(19) United States
(12) Patent Application Publication
(10) Pub. No.: US 2009/0078253 A1
(43) Pub. Date: Mar. 26, 2009

(54) Herbal and aromatherapy vaporizer

(76) Inventor: Fan Bao, City of Industry, CA (US)

Correspondence Address: DAVID AND RAYMOND PATENT FIRM, 108 N. YNEZ AVE., SUITE 128, MONTEREY PARK, CA, 91754, US

(21) Appl. No.: 11/903,424

(22) Filed: Sep. 20, 2007

(51) Int. Cl. A61M 16/10

(52) U.S. Cl. 128/203.26

(57) ABSTRACT

An herbal and aromatherapy vaporizer is arranged to direct hot air flow moving longitudinally to retain the hot air contacting the treatment element for a longer period of time, and the hot air carries away more functional constituents from the treatment element to increase the vapor concentration. The vaporizer includes a heat generator, a vapor outlet, a treatment crucible and an inhaler unit all aligned longitudinally. The air is heated in the heat generator and exited at the vapor outlet, and then longitudinally passes through the treatment element in the treatment crucible. The treatment element is heated to release functional constituents which are carried away by the hot air to subsequently pass through the inhaling guider at the exhausting end thereof. The vapor is collected by an inflatable pocket at the exhausting end and the user inhales the vapor at an opposed mouthpiece end of the inhaling guider.

### BACKGROUND OF THE INVENTION

1. Field of Invention

The present invention is to provide a vaporizer, and more particularly to an herbal and aromatherapy vaporizer, wherein the constituents thereof include a receiving compartment, a vapor outlet, a vaporizing device, a treatment filler and an inhaler unit which are arranged in a longitudinal manner to enhance the vapor contacting with the treatment element.

2. Description of Related Arts

A vaporizer is a device commonly used for releasing medicinal or psychoactive compounds from plant materials, such as tobacco. For medical or recreational purpose, vaporizing is an alternative to smoking. Rather than burning, a vaporizer heats the plant material to around 200° C. so that the volatile psychoactive and medicinal constituents contained in the plant melt and phase into an aromatic vapor that does not contain the particular matter, such as tars, found in the smoke.

Generally speaking, the substance can be heated by the vaporizer by means of convection or conduction. As to conduction heating, the substance is placed on a metal plate or a hot surface which is heated to release the active constituents. The conduction heating vaporizer is disadvantageous because of direct contact between hot surface and the herbs or plant materials which may be burned as a result.

On the other hand, the herbs or plant materials do not directly contact with the heating element in convection heating vaporizers. Instead, hot air flow passes through medicinal or psychoactive substances, heating them rapidly and allowing the release of the active constituents. Comparing to conduction heating, convection heating can release more active constituents. The released vapor can be subsequently stored in a glass dome or plastic bag for later inhalation.

In the U.S. Pat. No. 6,513,524, Storz disclosed a vaporizer in which hot air is vertically blown through the plant material and the aroma- and active substances are transitioned into vapor which is collected into a balloon via a valve. The vapor can then be inhaled out by the user through an external add-on mouthpiece connected to the valve. However, this invention is disadvantageous in several aspects.

In Storz, the heat chamber, air flow tube, herb container, and the inhaling portion are all vertically aligned. The air from the air inlet is heated in the heat chamber and according to the principles of physics, the heated air has lower density and diffuses more freely than the air before heated. More specifically, the heated air moves faster and has a strong tendency to move upwards after leaving the heat chamber such that when the heated air passes through the container with the herbs, the retention time for the heated air to stay on the herbs is shorter. In other words, the heated air does not have enough time to contact the herbs and carry away the constituents released therefrom because it moves so fast and upwardly. Thus, the concentration of the constituents in the vapor may not be enough to serve some recreational or medicinal purposes.

When the heated air passes through the valve to the vapor balloon, an upward lifting force is thus generated due to the strong upward momentum of the heated air such that the lifting force may cause the valve and balloon set to dislocate from the receptacle, which may result in vapor leaking and thus reduce the effectiveness of the vaporizer. Also, the buoyancy of the air in the balloon can provide another lifting force to worsen this situation. Therefore, Storz must provide a seat to hold the receptacle.

Furthermore, when the heated air leaves the heat chamber, it has to pass through an air flow tube before reaching the herbs in the container. Heat loss is resulted along the path of air flow and the longer the air flow tube, the more heat loss. In Storz, when the user wants to inhale the vapor stored in the vapor balloon, a mouth piece has to be mounted to the valve set of the vaporizer, meaning the mouth piece is not built-in to the vaporizer. It is thus inconvenient to the user to carry a separate mouth-piece when he wants or needs to inhale the vapor in the vapor balloon. By the way, the temperature regulator and the on/off switch discovered in Storz are commonly used in conventional vaporizers.

### SUMMARY OF THE PRESENT INVENTION

A main object of the present invention is to provide an herbal and aromatherapy vaporizer, wherein the air flow is arranged to move longitudinally to increase the contact between the heated air and the treatment element, and thus the constituents of the herbs can be effectively carried by the heated air.

Another object of the present invention is to provide an herbal and aromatherapy vaporizer, wherein the air flow is arranged to move longitudinally to minimize the lifting force due to the upward momentum of the heated air and consequently avoid vapor leaking caused by dislocation between the constituents of the vaporizer because of the lifting force.

Another object of the present invention is to provide an herbal and aromatherapy vaporizer, wherein an air transportation distance is shorter such that the heat loss is minimized.

Another object of the present invention is to provide an herbal and aromatherapy vaporizer, wherein the mouth-piece is built-in and it is convenient for the user to inhale the vapor stored in the balloon.

Accordingly, in order to accomplish the above objects, the present invention provides an herbal and aromatherapy vaporizer, comprising:

a housing having a receiving compartment and a vapor outlet facing sidewardly;

a vaporizing device comprising a heat generator supported in the receiving compartment and an air inlet arranged in such a manner that the heat generator is adapted for heating up an air flow entrancing from the air inlet to generate a hot air flow exiting at the vapor outlet at a longitudinal direction;

a treatment filler comprising a treatment crucible detachably coupled with the housing at the vapor outlet thereof, wherein the treatment crucible comprises a crucible body having a treatment cavity for receiving a treatment element therein and a crucible cover enclosing the treatment cavity, such that the hot air flow longitudinally passes through the treatment cavity from the vapor outlet to form a treatment vapor; and

an inhaler unit comprising a tubular inhaling guider longitudinally supported by the housing, wherein the inhaling guider has a mount-piece end communicatively coupling with the treatment filler and an opposed exhausting end for exhausting the treatment vapor.

These and other objectives, features, and advantages of the present invention will become apparent from the following detailed description, the accompanying drawings, and the appended claims.

### BRIEF DESCRIPTION OF THE DRAWINGS

FIG. 1 is a schematic view of the herbal and aromatherapy vaporizer according to a preferred embodiment of the present invention.

FIG. 2 is a sectional view of the housing and receiving compartment of the vaporizer according to the above preferred embodiment of the present invention.

FIG. 3 is a sectional view of the treatment filler of the vaporizer according to the above preferred embodiment of the present invention.

FIG. 4A is a sectional view of the inhaling guider in its opening position according to the above preferred embodiment of the present invention.

FIG. 4B is a sectional view of the inhaling guider in its closed position according to the above preferred embodiment of the present invention.

FIG. 5 is a sectional view of the longitudinal air flow path in the vaporizer according to the above preferred embodiment of the present invention.

FIG. 6 is a schematic representation illustrating the user inhaling the treatment vapor from the built-in mouth piece according to the above preferred embodiment of the present invention.

### DETAILED DESCRIPTION OF THE PREFERRED EMBODIMENT

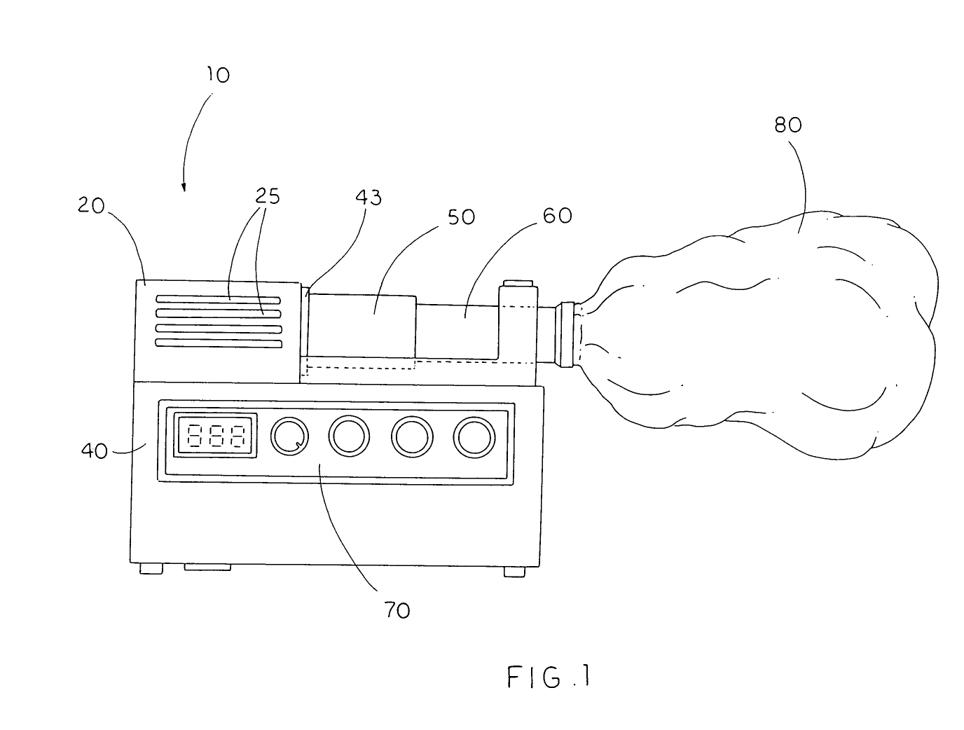

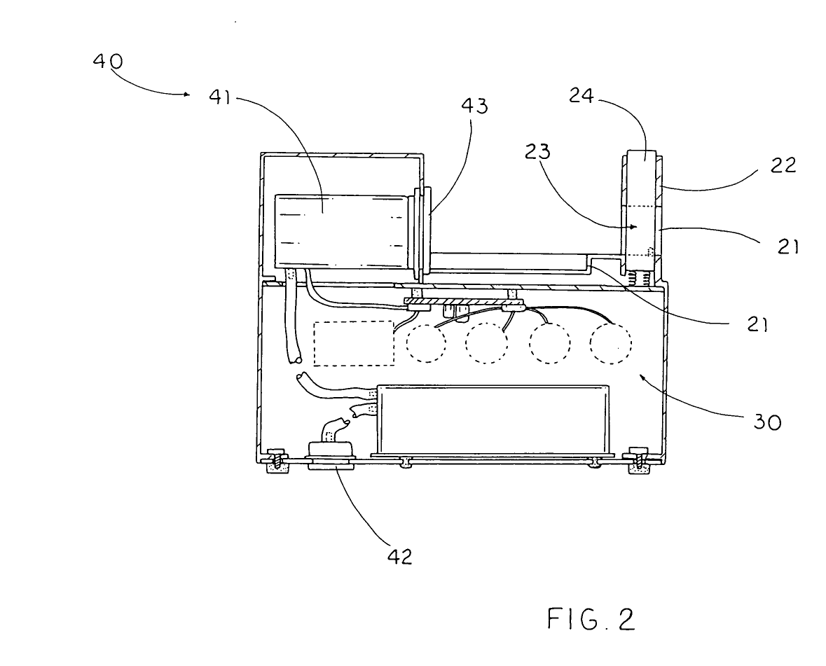

Referring to FIG. 1 and FIG. 2, an herbal and aromatherapy vaporizer 10 according to a preferred embodiment of the present invention is illustrated, wherein herbal and aromatherapy vaporizer 10 comprises a housing 20 and a vaporizing device 40.

The housing 20 has a receiving compartment 30, an air inlet 42 and vapor outlet 43, wherein when air is sucked from the air inlet 42, the vaporizing device 40 is arranged to heat up the air to form a flow of vapor being exited at the vapor outlet 43. Accordingly, the air inlet 42 is provided at the bottom side of the housing 20 while the vapor outlet 43 is provided at an upper portion of the housing 20 and is facing sidewardly to guide the vapor (hot air) flowing at a longitudinal direction.

The vaporizing device 40 comprises a heat generator 41 communicating with the air inlet 42 and arranged to heat up the air flow entrancing from the air inlet 42. It is worth to mention that the heat generator 41 is located at the upper portion of the housing 20 to generate the hot air flow existing at the vapor outlet 43 at a longitudinal direction.

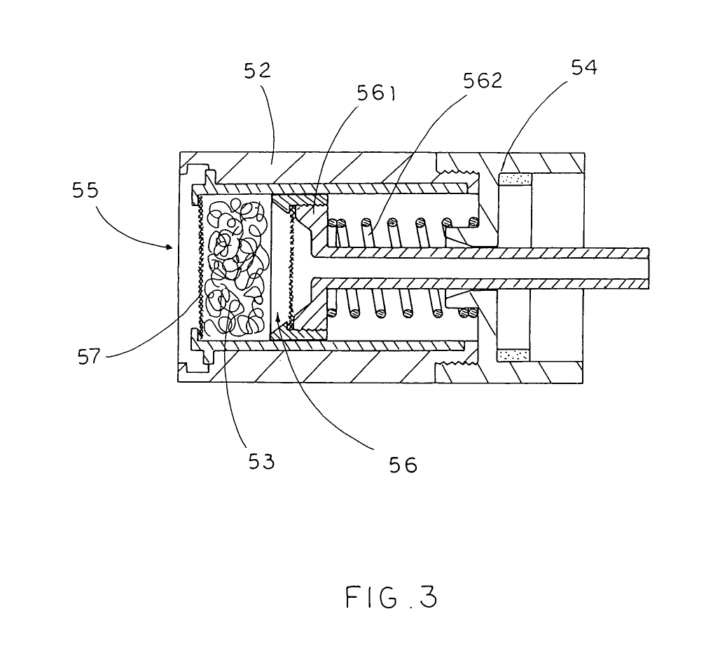

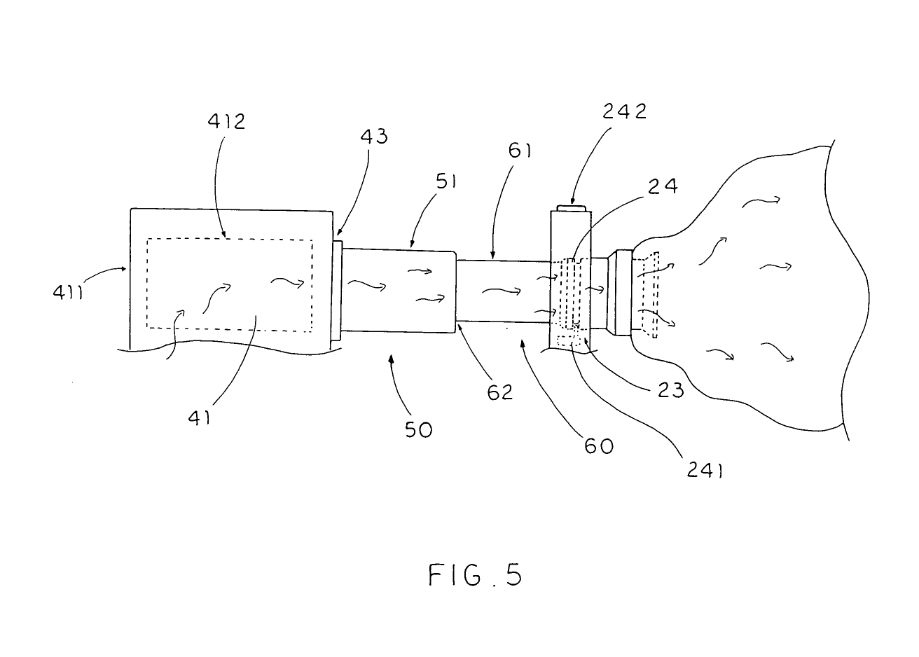

The vaporizer 10 further comprises a treatment filler 50, an inhaler unit 60 and a control panel 70. The treatment filler 50 comprises a treatment crucible 51 detachably coupled with the housing at the vapor outlet 43. The treatment crucible 51 has a crucible body 52 with a treatment cavity 53 for receiving a treatment element therein and a crucible cover 54 enclosing the treatment cavity 53. The bottom 55 of the treatment crucible is made of a screen mesh 57, such that the hot air flow from the vapor outlet 43 can longitudinally passes through the screen mesh 57 to the treatment cavity 53, contacting the treatment element to form a treatment vapor, as shown in FIG. 3 and FIG. 5.

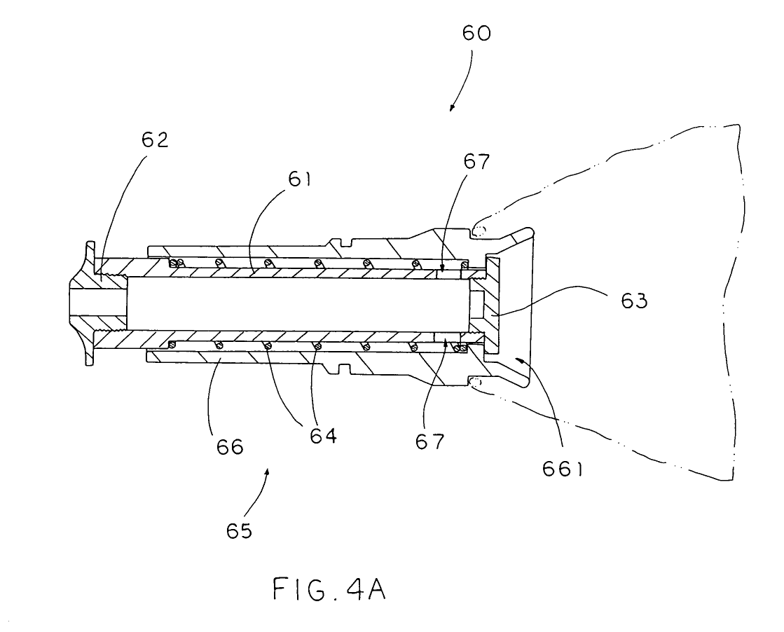

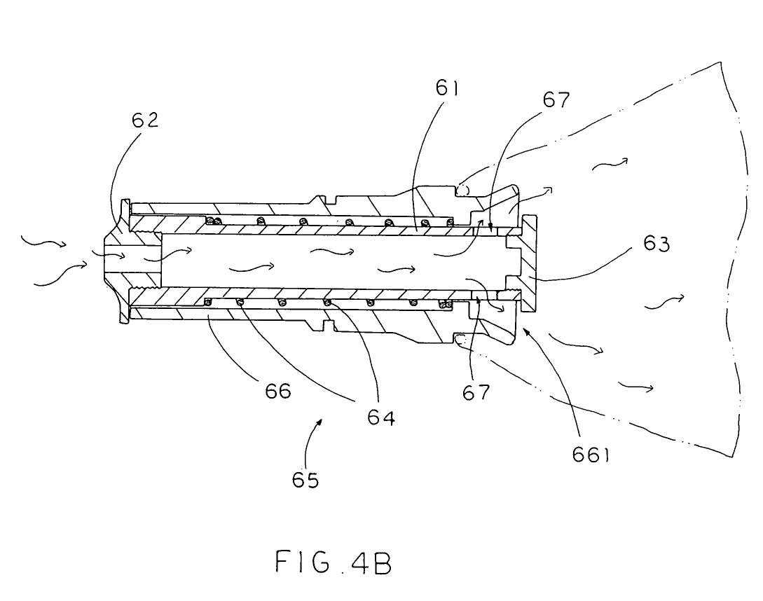

As can be seen in FIGS. 4A and 4B, the inhaler unit 60 comprises a tubular inhaling guider 61 longitudinally supported by the housing 20. One end of the inhaler guider 61 is a mouth-piece end 62 communicatively and longitudinally coupling with the treatment filler 50 and an opposed exhausting end 63 for exhausting the treatment vapor. In short, the heat generator 41, the vapor outlet 43, the treatment crucible 51 and the inhaler unit 60 are all aligned longitudinally.

Accordingly, the air is heated in the heat generator 41 and exits at the vapor outlet 43, and then longitudinally passes through the screen mesh 57 of the treatment crucible 51 and the treatment element therein. The treatment element is heated and releases functional constituents which are carried away by the hot air which subsequently passes through the tubular inhaling guider 61 to the exhausting end 63.

Referring to FIG. 2 and FIG. 5, the housing 20 further comprises a supporting frame 21 for horizontally supporting the inhaler unit 60 in position. The supporting frame 21 has a supporting wall 22 upwardly extended from the housing 20 and a locker slot 23 coaxially aligned with the vapor outlet 43, such that when the inhaler unit 60 is slidably passing through the locker slot 23, the mouth-piece end 62 of the inhaling guider 61 is coupled at the vapor outlet 43 through the treatment filler 50.

The supporting frame 21 further comprises a releasable locker 24 releasably locking the inhaler unit 60 at the locker slot 23. The releasable locker 24 comprises a locking arm 241 movably coupling at the locker slot 23 to lock up the inhaler unit 60 and a locking button 242 movably mounted at the supporting frame 21. When the locking button 242 is pressed down, the locking arm 241 correspondingly moves down to release the inhaler unit 60 so as to allow the inhaler unit 60 being slidably detached from the supporting frame 21.

The control panel 70 comprises a temperature regulator, on/off switches for heat, air and power, connected to a circuit board located in the receiving compartment 30. When the vaporizer is turned on, the air enters the vaporizer from the air inlet 42 and is transported through the tubing in the housing 20 to the heat generator 41. The heat generator 41 is located longitudinally at the upper portion of the housing, wherein one end serves as a receiving end 44 adapted for receiving the air from the air inlet and the other end is the vapor outlet 43 from which the heated air exits at a longitudinal direction.

The heat generator 41 has a heat chamber 411 longitudinally supported in the housing 20 at a position adjacent to the vapor outlet 43 and a heating element 412 supported in the heat chamber 411 for heating up the air flow within the heat chamber 411, so as to minimize a distance between the heat chamber 411 and the treatment crucible 51. In other words, heat loss can be reduced by minimizing the heat transportation distance between the heat chamber 411 and the treatment crucible 51. In addition, the heat generator 41 is surrounded by a plurality of venting slots 25 to provide an outlet for excessive heat and enhance heat generating efficiency of the heat generator 41, as shown in FIG. 1 and FIG. 5.

The hot air then enters the treatment crucible 51 of the treatment filler 50 and passes through the treatment element. A treatment presser 56 coupled with the treatment crucible 51 applies a predetermined pressing force against the treatment element in a packed manner, such that the treatment element can be evenly distributed in the treatment cavity 53 and the hot air can more effectively carry away the functional constituents when it passes through.

Furthermore, the treatment presser 56 comprises a pressing member 561 slidably disposed in the treatment cavity 53 and a resilient element 562 with two ends biased against the pressing member 561 and the crucible cover 54 respectively. The resilient element 562 applies the pressing force against the pressing member 561 to press against the treatment element in a packed manner in the treatment cavity 53. By applying different magnitude of pressing force, the resilient element 562 can keep the treatment element in different amount in the treatment cavity 53 in a packed manner, as can be seen in FIG. 3. It is worth to mention that since the treatment crucible 51 is supported longitudinally, the treatment presser 56 can retain the treatment element close to the vapor outlet 43.

Unlike the air flow path designed in a vertical manner illustrated previously, the hot air enters the treatment filler 50 longitudinally, such that the hot air with lower density and strong upward tendency can be confined and retained in the treatment cavity 53 for a longer period of time wherein the hot air has more contact with the treatment element and thus carries away more functional constituents thereof. In other words, the functional constituents can be more effectively collected in the present invention than in the vaporizer with vertical air flow path.

After collecting the functional constituents from the treatment element, the treatment vapor longitudinally flows into the inhaling guider 61 of the inhaler unit 60 which further comprises a valve control 65 for controlling the treatment vapor passing through the inhaling guider 61. The valve control 65 has an outer sliding sleeve 66 coaxially and slidably coupling with the inhaling guider 61 to slide between an opened position and a closed position as shown in FIGS. 4A and 4B.

A resilient retainer 64 in the inhaling guider 61 is adapted for applying a resilient force to retain the inhaling guider 61 at the closed position in which the sliding sleeve 66 is slid to enclose the exhausting end 63 of the inhaling guider 61, while at the opening position, the sliding sleeve 66 is slid towards the mouth-piece end 62 of the inhaling guider 61 to expose the exhausting end 63 for allowing the treatment vapor exhausting therefrom. Accordingly, when the inhaling guider 61 is detached from the housing 20, the sliding sleeve 66 is automatically slid to enclose exhausting end 63 of the inhaling guider 61 for prevent the treatment vapor from being leakage.

The inhaling guider 61 also includes a plurality of vapor vents 67 spacedly provided at the outer circumferential surface of the inhaling guider 61 at the exhausting end 63 for outwardly and radially exhausting the treatment vapor when the valve control 65 is in the opening position.

The outer sliding sleeve 66 further comprises an actuation ring 661 radially and outwardly protruded from an outer circumferential surface of the sliding sleeve 66. When the inhaler unit 60 slidably passes through the locker slot 23 to couple the mouth-piece end 62 with the treatment filler 50, the actuation ring 661 is blocked by the supporting frame 21 to ensure the sliding sleeve 66 being slid at the opening position so as to allow the treatment vapor flowing to the exhausting end 63.

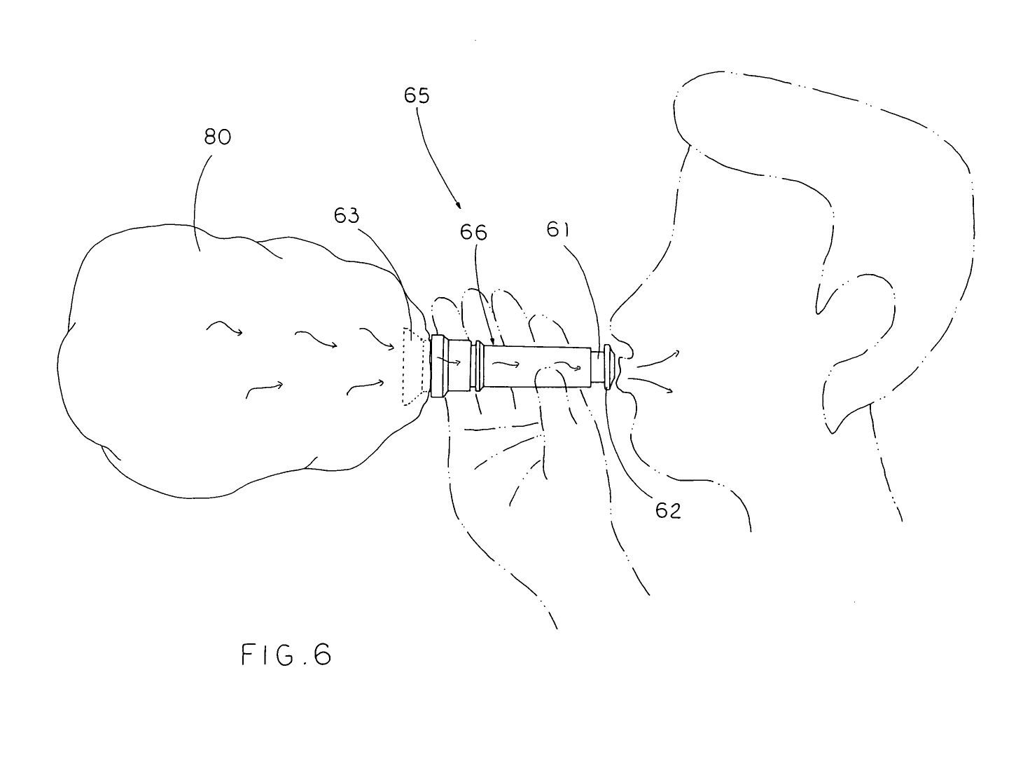

Referring to FIG. 6, an inflatable pocket 80 with a pocket opening is detachably coupled at the exhausting end 63 of the inhaling guider 61 for collecting the treatment vapor which flows into the inflatable pocket 80 when the valve control 65 is at the opening position. More specifically, when the treatment filler 50 longitudinally aligns with the inhaler unit 60 at the upper portion of the housing 20 of the vaporizer 10, the mouth-piece end 62 is detachably and longitudinally coupled with the crucible cover 54, wherein the crucible cover 54 is designed to press the sliding sleeve 66 slide towards the mouth-piece end 62 of the inhaling guider 61 to expose the exhausting end 63 for allowing the treatment vapor exhausting to the inflatable pocket 80.

When the inhaling guider 61 is detached from the housing 20, the inhaling guider 61 is adapted for a user to inhale the treatment vapor in the inflatable pocket 80 via the mouth-piece end 62 of the inhaling guider 61. It is worth to mention that when the inhaling guider 61 is detached from the housing 20, the sliding sleeve 66 is automatically slid to enclose exhausting end 63 of the inhaling guider 61 for prevent the treatment vapor being leakage from the inflatable pocket 80.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An herbal and aromatherapy vaporizer, comprising:
a housing having a receiving compartment and a vapor outlet facing sidewardly;
a vaporizing device comprising a heat generator supported in said receiving compartment and an air inlet arranged in such a manner that said heat generator is adapted for heating up an air flow entrancing from said air inlet to generate a hot air flow exiting at said vapor outlet at a longitudinal direction;
a treatment filler comprising a treatment crucible detachably coupled with said housing at said vapor outlet thereof, wherein said treatment crucible comprises a crucible body having a treatment cavity for receiving a treatment element therein and a crucible cover enclosing said treatment cavity, such that said hot air flow longitudinally passes through said treatment cavity from said vapor outlet to form a treatment vapor; and
an inhaler unit comprising a tubular inhaling guider longitudinally supported by said housing, wherein said inhaling guider has a mount-piece end communicatively coupling with said treatment filler and an opposed exhausting end for exhausting said treatment vapor.

2. The herbal and aromatherapy vaporizer, as recited in claim 1, wherein said treatment filler further comprises a treatment presser coupling with said treatment crucible for applying a predetermined pressing force against said treatment element in a packed manner, wherein said treatment presser comprises a pressing member slidably disposed in said treatment cavity and a resilient element having two ends biasing against said pressing member and said crucible cover respectively for applying said pressing force against said pressing member to press against said treatment element in said treatment cavity.

3. The herbal and aromatherapy vaporizer, as recited in claim 1, further comprising an inflatable pocket having a pocket opening detachably coupling at said exhausting end of said inhaling guider for collecting said treatment vapor such that when said inhaling guider is detached from said housing, said inhaling guider is adapted for a user to inhale said treatment vapor in said inflatable pocket via said mount-piece end of said inhaling guider.

4. The herbal and aromatherapy vaporizer, as recited in claim 2, further comprising an inflatable pocket having a pocket opening detachably coupling at said exhausting end of said inhaling guider for collecting said treatment vapor such that when said inhaling guider is detached from said housing, said inhaling guider is adapted for a user to inhale said treatment vapor in said inflatable pocket via said mount-piece end of said inhaling guider.

5. The herbal and aromatherapy vaporizer, as recited in claim 1, wherein said inhaler unit further comprises a valve control for controlling said treatment vapor passing through said inhaling guider, wherein said valve control comprises an outer sliding sleeve coaxially and slidably coupling with said inhaling guider to slide between an opened position and a closed position, and a resilient retainer coupling between said inhaling guider for applying a resilient force therebetween to retain said inhaling guider at said closed position, wherein at said opening position, said sliding sleeve is slid towards said mount-piece end of said inhaling guider to expose said exhausting end for allowing said treatment vapor exhausting therefrom, and at said closed position, said sliding sleeve is slid to enclose said exhausting end of said inhaling guider.

6. The herbal and aromatherapy vaporizer, as recited in claim 2, wherein said inhaler unit further comprises a valve control for controlling said treatment vapor passing through said inhaling guider, wherein said valve control comprises an outer sliding sleeve coaxially and slidably coupling with said inhaling guider to slide between an opened position and a closed position, and a resilient retainer coupling between said inhaling guider for applying a resilient force therebetween to retain said inhaling guider at said closed position, wherein at said opening position, said sliding sleeve is slid towards said mount-piece end of said inhaling guider to expose said exhausting end for allowing said treatment vapor exhausting therefrom, and at said closed position, said sliding sleeve is slid to enclose said exhausting end of said inhaling guider.

7. The herbal and aromatherapy vaporizer, as recited in claim 4, wherein said inhaler unit further comprises a valve control for controlling said treatment vapor passing through said inhaling guider, wherein said valve control comprises an outer sliding sleeve coaxially and slidably coupling with said inhaling guider to slide between an opened position and a closed position, and a resilient retainer coupling between said inhaling guider for applying a resilient force therebetween to retain said inhaling guider at said closed position, wherein at said opening position, said sliding sleeve is slid towards said mount-piece end of said inhaling guider to expose said exhausting end for allowing said treatment vapor exhausting therefrom, and at said closed position, said sliding sleeve is slid to enclose said exhausting end of said inhaling guider.

8. The herbal and aromatherapy vaporizer, as recited in claim 5, wherein said inhaling guider further has a plurality of vapor vents spacedly provided at an outer circumferential surface of said inhaling guider at said exhausting end thereof for outwardly and radially exhausting said treatment vapor, wherein said sliding sleeve is slid to cover said vapor vents at said closed position.

9. The herbal and aromatherapy vaporizer, as recited in claim 6, wherein said inhaling guider further has a plurality of vapor vents spacedly provided at an outer circumferential surface of said inhaling guider at said exhausting end thereof for outwardly and radially exhausting said treatment vapor, wherein said sliding sleeve is slid to cover said vapor vents at said closed position.

10. The herbal and aromatherapy vaporizer, as recited in claim 7, wherein said inhaling guider further has a plurality of vapor vents spacedly provided at an outer circumferential surface of said inhaling guider at said exhausting end thereof for outwardly and radially exhausting said treatment vapor, wherein said sliding sleeve is slid to cover said vapor vents at said closed position.

11. The herbal and aromatherapy vaporizer, as recited in claim 1, wherein said housing comprises a supporting frame for horizontally supporting said inhaler unit in position, wherein said supporting frame comprises a supporting wall, which is upwardly extended from said housing, has a locker slot coaxially aligned with said vapor outlet such that when said inhaler unit is slidably passing through said locker slot, said mount-piece end of said inhaling guider is coupled at said vapor outlet through said treatment filler.

12. The herbal and aromatherapy vaporizer, as recited in claim 4, wherein said housing comprises a supporting frame for horizontally supporting said inhaler unit in position, wherein said supporting frame comprises a supporting wall, which is upwardly extended from said housing, has a locker slot coaxially aligned with said vapor outlet such that when said inhaler unit is slidably passing through said locker slot, said mount-piece end of said inhaling guider is coupled at said vapor outlet through said treatment filler.

13. The herbal and aromatherapy vaporizer, as recited in claim 10, wherein said housing comprises a supporting frame for horizontally supporting said inhaler unit in position, wherein said supporting frame comprises a supporting wall, which is upwardly extended from said housing, has a locker slot coaxially aligned with said vapor outlet such that when said inhaler unit is slidably passing through said locker slot, said mount-piece end of said inhaling guider is coupled at said vapor outlet through said treatment filler.

14. The herbal and aromatherapy vaporizer, as recited in claim 11, wherein said supporting frame further comprises a releasable locker releasably locking said inhaler unit at said locker slot, wherein said releasable locker comprises a locking arm movably coupling at said locker slot to lock up said inhaler unit and a locking button movably mounted at said supporting frame to move said locking arm out of said locker slot to release said inhaler unit so as to allow said inhaler unit being slidably detached from said supporting frame.

15. The herbal and aromatherapy vaporizer, as recited in claim 12, wherein said supporting frame further comprises a releasable locker releasably locking said inhaler unit at said locker slot, wherein said releasable locker comprises a locking arm moveably coupling at said locker slot to lock up said inhaler unit and a locking button movably mounted at said supporting frame to move said locking arm out of said locker slot to release said inhaler unit so as to allow said inhaler unit being slidably detached from said supporting frame.

16. The herbal and aromatherapy vaporizer, as recited in claim 13, wherein said supporting frame further comprises a releasable locker releasably locking said inhaler unit at said locker slot, wherein said releasable locker comprises a locking arm moveably coupling at said locker slot to lock up said inhaler unit and a locking button movably mounted at said supporting frame to move said locking arm out of said locker slot to release said inhaler unit so as to allow said inhaler unit being slidably detached from said supporting frame.

17. The herbal and aromatherapy vaporizer, as recited in claim 13, wherein said sliding sleeve further has an actuation ring radially and outwardly protruded from an outer circumferential surface of said sliding sleeve and arranged in such a manner that when said inhaler unit is slidably passing through said locker slot to couple said mount-piece end of said inhaling guider with said treatment filler, said actuation ring is blocked by said supporting frame to ensure said sliding sleeve being slid at said opening position.

18. The herbal and aromatherapy vaporizer, as recited in claim 16, wherein said sliding sleeve further has an actuation ring radially and outwardly protruded from an outer circumferential surface of said sliding sleeve and arranged in such a manner that when said inhaler unit is slidably passing through said locker slot to couple said mount-piece end of said inhaling guider with said treatment filler, said actuation ring is blocked by said supporting frame to ensure said sliding sleeve being slid at said opening position.

19. The herbal and aromatherapy vaporizer, as recited in claim 1, wherein said heat generator comprises a heat chamber supported in said housing at a position adjacent to said vapor outlet and a heating element supported in said heat chamber for heating up said air flow within said heat chamber, so as to minimize a distance between said heat chamber and said treatment crucible.

20. The herbal and aromatherapy vaporizer, as recited in claim 7, wherein said heat generator comprises a heat chamber supported in said housing at a position adjacent to said vapor outlet and a heating element supported in said heat chamber for heating up said air flow within said heat chamber, so as to minimize a distance between said heat chamber and said treatment crucible.

21. The herbal and aromatherapy vaporizer, as recited in claim 18, wherein said heat generator comprises a heat chamber supported in said housing at a position adjacent to said vapor outlet and a heating element supported in said heat chamber for heating up said air flow within said heat chamber, so as to minimize a distance between said heat chamber and said treatment crucible.
